(19) United States
(12) Patent Application Publication
(10) Pub. No.: US 2004/0033614 A1
(43) Pub. Date: Feb. 19, 2004

(54) Detection of oxidative intermediates in biological samples

(76) Inventor: Rajiv Agarwal, Carmel, IN (US)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/437,684

(22) Filed: May 14, 2003

Related U.S. Application Data
(60) Provisional application No. 60/385,356, filed on May 31, 2002.

(51) Int. Cl. G01N 33/00

(52) U.S. Cl. 436/128

(57) ABSTRACT

Methods are provided for detecting oxidative intermediates in biological samples. These methods may be used to advantage to assess oxidative kidney injury and mitigation of kidney injury in response to drugs, diet and other therapies in patients with chronic kidney disease.

### CROSS REFERENCE TO RELATED APPLICATION

This application claims priority to U.S. provisional application 60/385,356 filed May 31, 2002, the entire disclosure of which is incorporated by reference herein.

### FIELD OF THE INVENTION

The present invention pertains to methods for monitoring oxidative intermediates in biological samples. More specifically, methods are provided to monitor the urinary presence of carbonylated proteins and lipids. Detection of carbonylated proteins and lipids in urine can be used to advantage as a diagnostic tool for assessing kidney injury as well as the efficacy of drugs, diet or other therapies administered to treat kidney injury in patients with chronic kidney disease.

### BACKGROUND OF THE INVENTION

Several publications and patent documents are referenced in this application by full citations or by numerals in parentheses in order to more fully describe the state of the art to which this invention pertains. Full citations for these references are found at the end of the specification. The disclosure of these publications and patents are incorporated by reference herein.

Each year in the United States, more than 50,000 people are diagnosed with end-stage renal disease (ESRD), a serious condition in which the kidneys fail to rid the body of wastes. ESRD is the final stage of a slow deterioration of the kidneys, a process known as nephropathy. Diabetes is the most common cause of ESRD, resulting in approximately one-third of new ESRD cases, and high blood pressure is the second leading cause of ESRD, accounting for nearly 30 percent of all cases. As a result, elevated blood pressure and severe proteinuria resulting from diabetes are major predictors of kidney disease (1). However, the mechanism(s) in which proteinuria results in ESRD is not completely understood (2).

One paradigm implicates proteinuria as a mediator of tubulointerstitial damage based on the observations that the blood protein, albumin, can stimulate the production of proinflammatory cytokines in proximal tubular cells via activation of the redox sensitive gene, nuclear factor kappa B (3, 4). Furthermore, chemokine expression in the kidney is modulated by the redox state, which in turn is modulated by angiotensin II, a protein regulator of blood pressure. Current therapies for kidney disease target the reduction of blood pressure and proteinuria. However, they do not address direct treatment of kidney damage.

Oxidative stress has emerged as a major pathophysiologic mechanism in mediating various disease states, including chronic kidney disease (5). For example, the formation of lipid hydroperoxides by oxidative lipid damage leads to dysfunction of membrane-bound receptors, and these compounds possess cytotoxic and mutagenic properties which are thought to play a major role in ageing and atherosclerosis (6). In addition, oxidation of polyunsaturated fatty acids (PUFA) in lipoproteins leads to formation of hydro- and endo-peroxides, which undergo fragmentation to yield a broad range of reactive intermediates including alkanals, alkenals, hydroxylalkenals and malondialdehyde (MDA) (7). These carbonyl compounds react with nucleophilic groups on proteins, resulting in chemical modification of the proteins.

It is not known if the oxidative stress and the proinflammatory state in the kidney can be favorably influenced without the reduction in blood pressure or proteinuria. Such demonstration can be of significant importance as the current therapies for chronic kidney disease address the treatment of high blood pressure and the reduction of proteinuria, but not the direct treatment of renal damage caused by oxidative stress.

### SUMMARY OF THE INVENTION

In accordance with the present invention, methods for detecting carbonylated proteins or lipids in a biological sample as biomarkers for oxidative tissue damage are provided. In a preferred embodiment, carbonylated proteins and lipids are isolated from urine as biomarkers for oxidative damage to the kidney. Levels of such carbonylated proteins and lipids are determined before, during and after administration of therapeutic agents for the treatment of chronic kidney disease to assess the efficacy of such agents. A reduction in the levels of carbonylated proteins and lipids is indicative of the efficacy of these therapeutic agents.

In a preferred embodiment, carbonylated proteins and malondialdehyde are detected in the urine using HPLC methods.

In another embodiment of the invention, a kit is provided for detecting oxidative kidney injury in a patient suspected of having kidney disease.

### BRIEF DESCRIPTION OF THE DRAWINGS

FIG. 1 shows two chromatograms, one obtained from a 1,1,3,3-Tetraethoxypropane standard after derivatizing with thiobarbituric acid (TBA) (left), and another after derivatizing urine with TBA (right).

FIGS. 2A and 2B show two graphs illustrating the extraction efficiency of n-butanol (FIG. 2A) and ethyl acetate (FIG. 2B).

FIG. 3 shows a standard curve of malondialdehyde (MDA).

FIG. 4 shows a graph delineating a log-linear decline in concentration of MDA.

FIG. 5 shows Western blots of urine and plasma protein samples before and after administration of losartan. Each sample was run before and after derivatization with dinitrophenyl hydrazine (DNP).

FIGS. 6A and 6B show graphs illustrating the levels of urinary albumin and total protein oxidation (FIG. 6A) and plasma albumin and total protein oxidation (FIG. 6B) before and after losartan therapy.

FIG. 7 shows two graphs illustrating the levels of urinary MDA (right) and plasma MDA (left) levels before and after losartan therapy.

FIG. 8 shows a graph delineating a decrease in oxidized urinary albumin and urinary monocyte chemotactic protein-1 (MCP1) levels.

### DETAILED DESCRIPTION OF THE INVENTION

Oxidative stress plays an important role in causing progressive kidney disease. However, it is not known if the oxidative stress and the proinflammatory state of the kidney can be favorably influenced independently from treatments for high blood pressure and proteinuria. Angiotensin converting enzyme inhibitors and angiotensin receptor blockers, such as losartan, are two classes of drugs that are commonly used for the treatment of progressive kidney disease.

In this study, patients with chronic kidney disease were examined to determine the effects of add-on angiotensin II blockage (losartan therapy) on the oxidative stress and proinflammatory state of the kidney independent from treatments for reduction of blood pressure and proteinuria. Oxidative damage to lipids was monitored by a fluorometric HPLC assay for malondialdehyde (MDA), and oxidative damage to proteins was measured by carbonyl concentration by HPLC as well as Western blotting of urinary and plasma proteins. Kidney proinflammatory state was measured by urinary monocyte chemotactic protein 1 (MCPl) excretion rate.

The results provided hereinbelow demonstrate that oxidant damage to urinary protein and lipids can be reduced with angiotensin II blockade, which is independent of reduction to blood pressure and proteinuria. These data demonstrate that urinary measurements of markers for oxidative damage, both carbonyls and lipid hydroperoxides, are more sensitive than plasma measurements in patients with chronic kidney disease.

Thus, in accordance with the present invention, methods are provided for the rapid, sensitive and large throughput screening of oxidative intermediates in biological samples as biomarkers for oxidative tissue damage. Such methods may be used to advantage to facilitate the development of novel therapies targeting oxidative stress in animals and humans.

In a preferred embodiment of the invention, methods are provided for the detection of carbonylated proteins and lipids in urine to assess oxidative damage to the kidney in patients with chronic kidney disease. Levels of such carbonylated proteins and lipids are determined before, during and after administration of therapeutic agents, such as losartan, for the treatment of chronic kidney disease to assess the efficacy of such agents. A reduction in the levels of carbonylated proteins and lipids is indicative of the efficacy of these therapeutic agents.

Urinary presence of oxidized lipids, such as malondialdehyde (MDA), are measured using an enhanced HPLC method for detecting MDA comprising the following sample preparation steps: (1) Urine samples are treated with an antioxidant, preferably butylated hydroxytoluene, and thiobarbituric acid at an acidic pH; (2) The samples are then heat derivatized at 100° C. for 1 hour; and (3) The samples are then extracted with n-butanol and injected at 1 minute intervals onto the HPLC apparatus. This method is superior over existing chromatographic methods for estimation of MDA in biological samples because: (a) it is rapid (samples can be injected at 1 minute intervals); (b) it eliminates extreme extraction procedures, such as additional protein precipitation steps; and (c) it eliminates the need for excessive column cleaning.

Similarly, the urinary presence of carbonylated proteins is detected using a HPLC method comprising the following steps: (1) Urine samples are derivatized with dinitrophenyl hydrazine (DNP) in trifluoroacetic acid (TFA); (2) carbonylated proteins are measured on a size exclusion column to separate the derivatized carbonyl groups from the derivatizing agent; and (3) carbonyl groups are detected using a diode array detector.

In another embodiment of the invention, the detection of oxidative intermediates in biological samples methods may be used to advantage for titrating drugs administered for treating disease states. In a preferred embodiment, the detection of carbonylated proteins and lipids in urine may be utilized to determine the optimal dosing of losartan for treating patients with chronic kidney disease. Additional antioxidative therapy, such as homocysteine lowering with folic acid and B12 therapy, which can improve oxidative protein damage and reduce cytokine excretion in the urine, may also be examined using the methods of the invention.

In yet another embodiment of the invention, a kit is provided for detecting oxidative kidney injury in patients with kidney disease. The kit comprises an instructional material useful for conveying the use of the inventive method in diagnosing oxidative kidney injury in patients with kidney disease. As used herein, an “instructional material” includes a publication, a recording, a diagram, or any other medium of expression which can be used to communicate the usefulness of the inventive method for its designated use. The kit also comprises a tube or vial or the like for obtaining a sample of urine from a patient suspected of having oxidative kidney injury. The kit further comprises antioxidant reagents, such as butylated hydroxytoluene (BTH) and dinitrophenyl hydrazine (DNP), as well as thiobarbituric acid (TBA) and phosphoric acid solution. These reagents will be added to the urine sample in the collection tube or vial prior to laboratory analysis to screen for the presence of carbonylated proteins and lipids.

The following description sets forth the general procedures involved in practicing the present invention. To the extent that specific materials are mentioned, it is merely for purposes of illustration and is not intended to limit the invention. Unless otherwise specified, general biochemical and molecular biological procedures, such as those set forth in Sambrook et al., Molecular Cloning, Cold Spring Harbor Laboratory (1989) (hereinafter “Sambrook et al.”) or Ausubel et al. (eds) Current Protocols in Molecular Biology, John Wiley & Sons (1997) (hereinafter “Ausubel et al.”) are used.

I. Definitions:

The following definitions are provided to facilitate an understanding of the present invention:

“High-pressure (high-performance) liquid chromatography” or “HPLC” as used herein is a technique for rapid separation of solutes on a solid support, based on adsorption, ion-exchange, size exclusion, hydrophobic interaction chromatography or reverse-phase chromatography. “Reverse-phase HPLC” is a chromatographic technique which utilizes a nonpolar stationary phase and a polar eluent.

“Oxidative injury” as used herein refers to the damage to tissues and organs in animals and humans that results from the oxidation of proteins or lipids.

“Oxidative intermediates” as used herein refers to reactive intermediates that are formed as a result of the oxidation of lipids or proteins. Exemplary oxidative intermediates formed in response to the oxidation of lipids include, but without limitation, alkanals, alkenals, hydroxylalkenals and MDA. Oxidized proteins include, but without limitation, carbonylated proteins such as carbonylated albumin.

“Proteinuria” as used herein is a condition in which proteins leak from the kidney into the urine.

II. HPLC Devices:

An HPLC device, as described herein, typically includes at least the following components: a column, packed with a suitable stationary phase, a mobile phase, a pump for forcing the mobile phase through the column under pressure, and a detector for detecting the presence of compounds eluting off of the column.

Routine methods and apparatus for carrying out HPLC procedures are well known in the art and are described, for example, in the following references: J. Chromatography, 192:222-227 (1980), J Liquid Chromatography, 4:661-680 (1981), and J. Chromatography, 249:193-198 (1982), the disclosures of which are incorporated by reference herein.

Suitable stationary phases for the practice of the present invention are those in which the compound of interest elutes. Preferred columns are reverse phase columns, which may be natural (silica gel with alkyl chains of different lengths) or a synthetic crosslinked polymer (consisting of styrene and divinylbenzene). Suitable detection devices include, without limitation, mass spectrometers, fluorescence detectors and UV detectors.

The following examples provide illustrative methods of practicing the instant invention, and are not intended to limit the scope of the invention in any way.

### EXAMPLE I

Rapid Fluorometric HPLC Determination of Malondialdehyde in Biological Samples

Existing chromatographic methods for the estimation of malondialdehyde (MDA) often require extensive extraction procedures, column cleaning or specialized equipment. To overcome these drawbacks, a rapid and sensitive HPLC method has been developed as described hereinbelow for the determination of MDA in urine.

I. Materials and Methods: under pressure, and a detector for detecting the presence of compounds eluting off of the column.

Routine methods and apparatus for carrying out HPLC procedures are well known in the art and are described, for example, in the following references: J. Chromatography, 192:222-227 (1980), J Liquid Chromatography, 4:661-680 (1981), and J. Chromatography, 249:193-198 (1982), the disclosures of which are incorporated by reference herein.

Suitable stationary phases for the practice of the present invention are those in which the compound of interest elutes. Preferred columns are reverse phase columns, which may be natural (silica gel with alkyl chains of different lengths) or a synthetic crosslinked polymer (consisting of styrene and divinylbenzene). Suitable detection devices include, without limitation, mass spectrometers, fluorescence detectors and UV detectors.

The following examples provide illustrative methods of practicing the instant invention, and are not intended to limit the scope of the invention in any way.

### EXAMPLE I

Rapid Fluorometric HPLC Determination of Malondialdehyde in Biological Samples

Existing chromatographic methods for the estimation of malondialdehyde (MDA) often require extensive extraction procedures, column cleaning or specialized equipment. To overcome these drawbacks, a rapid and sensitive HPLC method has been developed as described hereinbelow for the determination of MDA in urine.

I. Materials and Methods:

The following materials and methods are provided to facilitate the practice of the present invention:

A. Reagent Preparation

Ethanol (95%) and HPLC grade methanol were purchased from Fisher Scientific (Fair Lawn, N.J.). All other chemicals were purchased from Sigma (St. Louis, Mo.). Chemical solutions were prepared using distilled deionized water unless otherwise indicated. Butylated hydroxytoluene (BHT) solution was prepared in 95% ethanol to a final concentration of 0.05% BHT. A 0.44 M phosphoric acid solution (H₃PO₄) was obtained by diluting 1 ml concentrated phosphoric acid to 100 ml final volume. 2-thiobarbituric acid (TBA) was dissolved in water at 50-55° C. to a final concentration of 42 mM. 40% ethanol solution was obtained by diluting 420 ml of 95% ethanol to final volume of 1000 ml.

B. Standards

Standards and quality control samples were prepared using 97% 1,1,3,3-Tetraethoxypropane (TEP) with the stock standard solution containing 5 μM TEP in 40% ethanol solution. Standards were prepared by serial dilution of the stock standard with ethanol solution to obtain final concentrations of 5, 4, 2, 1, 0.5, 0.25, 0.125, and 0 (blank) μM TEP. Standard curves were prepared for analysis each day, as well as the quality control samples, 0.125, 0.5 and 2.0 μM TEP.

C. Sample Preparation

Sample derivatization was carried out in 2 mL capacity plastic centrifuge tubes fitted with screw-on caps. To a 50 μl aliquot of sample or TEP standard, 50 μl BHT solution, 400 μl H₃PO₄ solution, and 100 μl TBA solution were added. Sample tubes were capped tightly, vortexed and then heated for 1 hour in a 100° C. dry bath incubator. Following heat derivatization, samples were placed in an ice-water (0° C.) water bath for 5 minutes to cool. 250 μl n-butanol was subsequently added to each vial for extracting the MDA-TBA complex. Tubes were vortexed for 5 minutes and then centrifuged for 3 minutes at 14,000× min⁻¹ to separate the two phases. Aliquots of 100 μl were removed from the n-butanol layer of each sample and placed in HPLC vials for analysis.

D. Chromatographic Apparatus and Conditions

The chromatographic system consisted of a Hewlett-Packard Chromatographic Series 1100 autosampler and isocratic pump and Hewlett-Packard model 1046A programmable fluorescence detector (Palo Alto, Calif., USA). The autosampler was programmed to inject 10 μl of each sample at 1-minute intervals. The pump flow rate was 1.0 ml/min with the mobile phase comprised of methanol-buffer (40:60, v/v). The buffer was 50 mM potassium monobasic phosphate (anhydrous) with an adjusted pH of 6.8 using 5 M potassium hydroxide. The fluorescence detector was set at an excitation wavelength of 515 nm and emission wavelength of 553 nm. The photomultiplier tube and lamp flash frequency were optimized to give the most signal to noise ratio. The column was a Hewlett-Packard Hypersil 5 ODS 100×4.6 mm with a 5 μ ODS guard column (Altech Associates, Deerfield, Ill.) placed in a column warmer set to 37° C. Peak areas were determined using a ChromJet integrator (Thermo Separation Products, San Jose, Calif.).

E. Assessment of Recovery, Extracting Agent and Adduct Stability

Increasing amounts of TEP (0, 0.5, 1 and 2 μmol/L) were supplemented in water or plasma specimen from a single patient and extracted with either ethyl acetate or n-butanol. N-butanol extraction was performed as described above. For ethyl acetate extraction, 500 μl of the solvent was added to the derivatized sample, vortexed, centrifuged and then the supernatant was transferred to a microcentrifuge tube. This was carried out three times, followed by vacuum evaporation of supernatant and reconstitution in water.

In order to evaluate MDA-TBA adduct stability, the standard was re-injected 2.3 hours following initial injection. Additional samples were injected at timed intervals to calculate the decay constant by first order reaction models.

F. Calculations

Standard curves were created by linear regression of peak area obtained by integration versus known concentrations of MDA. Concentrations of quality controls and unknown samples were estimated by applying the linear regression equation of the standard curve to the unknown sample peak-area.

The recovery of MDA was estimated by calculating the percent difference between known concentrations of MDA and that obtained by applying the linear regression equation ((observed−expected)×100%/expected concentration). Interday and intraday coefficients of variation were obtained by one-way analysis of variance at each of the three concentrations of MDA (0.125, 0.5 and 2 μmol/L). The limit of detection was calculated by the method of Anderson (8).

The decay rate was calculated using a first order decay model. The ratio of initial to re-injected standard curve slopes was log transformed and then divided by elapsed time to yield the elimination rate constant. Fitting a slope to timed injections of MDA confirmed the first order reaction of the decay.

G. Subjects

The study was approved by the Institutional Review Board for Human Subjects at Indiana University. Urine and plasma samples were obtained after written informed consent in 10 normotensive volunteers attending a Veterans Administration outpatient medicine clinic. Urine samples were collected without preservatives and plasma was collected in EDTA and frozen at −86° C. before analysis.

II. Results:

A. Chromatography

Sample preparation and chromatographic conditions were adapted to meet certain criteria. Specifically, MDA is bound to proteins (9, 10), and if a protein precipitation step is carried out prior to derivatization, there is a risk of losing the analyte of interest. Moreover, not removing proteins from derivatized samples, will either necessitate extensive column washing following each analysis or result in a short column life.

To overcome these drawbacks, protein was not precipitated and the samples were derivatized after addition of butylated hydroxytoluene to prevent de novo formation of MDA. The derivatized sample was then extracted with n-butanol. These adjustments to the sample preparation enabled the measurement of the total content of MDA in the samples without having to precipitate MDA from bound proteins or perform extensive column washing.

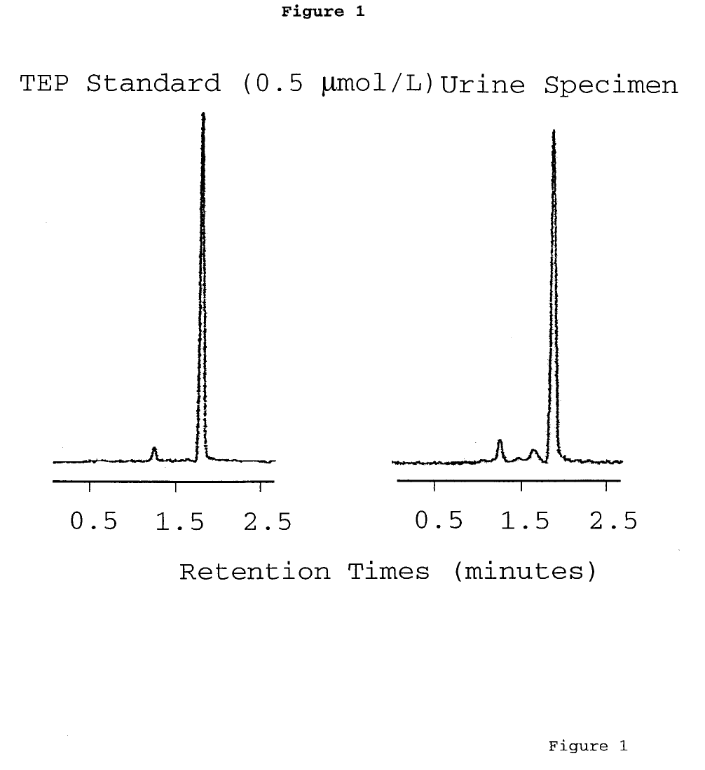

Chromatograms of a TEP standard and urine from a volunteer patient are shown in FIG. 1. Rapid injections of specimens were possible because of the short retention time and the absence of interfering peaks.

B. Linearity and Extraction Solvent

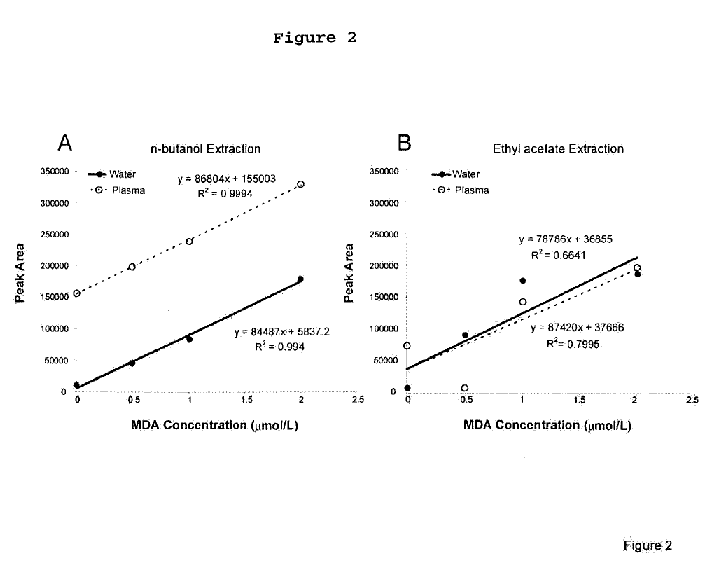

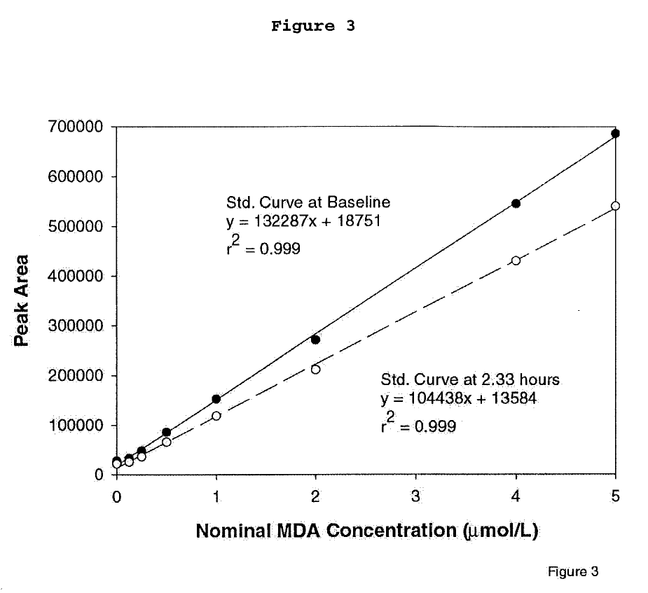

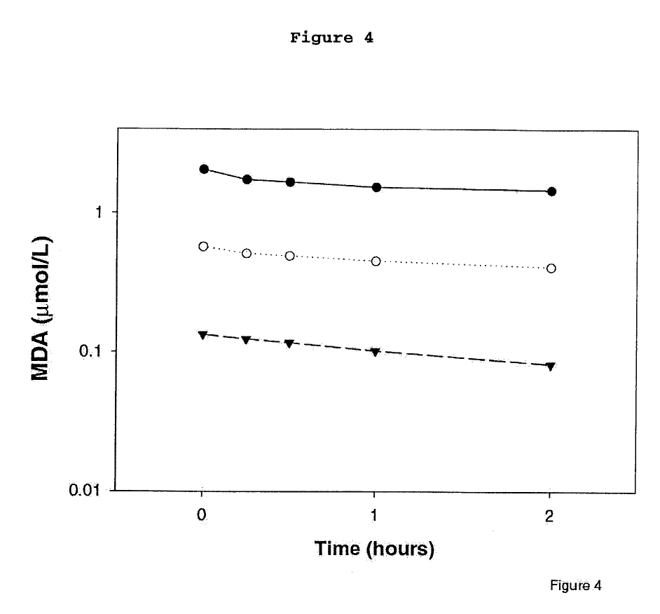

Calibration curves were created for 7 different concentrations of MDA (0-5 μM/L) by plotting the peak area versus the nominal MDA concentration. The correlation coefficient was 0.99 or better (FIG. 3). There was a small positive intercept noted in the blank sample, which has been reported by others. There was also a highly significant linear relationship between MDA concentration in water and plasma and peak area obtained by fluorescence (FIG. 2A). Furthermore, the slopes of peak area to MDA concentration were parallel in plasma and water when n-butanol was used as an extracting solvent (FIG. 2A). These results suggest that no matrix effect exists when n-butanol is used as an extracting solvent. However, plasma extracted with ethyl acetate did not have the expected positive intercept. Furthermore, ethyl acetate was not able to extract the MDA-TBA adduct efficiently from either plasma or water matrix. (FIG. 2B).

C. Recovery, Limit of Detection, and Intraday and Interday Coefficients of Variation

Within assay and between assays coefficients of variation for three different concentrations of MDA is reported in Table I.:

TABLE I
Recovery and Variability of Malondialdehyde Measurements

| | Intraday | Interday | |
|---|---|---|---|
| Concentration of | Coefficient of | Coefficient of | Recovery |
| MDA (μmol/L) | variation (%) | Variation (%) | (% ± SD %) |
| 0.125 | 10.3 | 4.6 | 99 ± 12 |
| 0.5 | 8.6 | 7.9 | 101 ± 9 |
| 2.0 | 10.8 | 3.6 | 88 ± 9 |

Within day variability was estimated between 8.6% and 10.3%. Between days variability was estimated between 3.6%-7.9%, and total recovery was between 88-101%. The limit of detection was measured as 0.128 μmol/L. Although a clear peak was noted at 0.125 μmol/L, a significant peak was also noted in the blank sample, thus increasing the limit of detection.

A decay in the MDA-TBA adduct was also observed (FIGS. 2A, 2B and 3). Upon re-injecting the same sample at timed intervals, or the entire standard curve at 2 to 3 hours, a first order breakdown was observed. From the standard curve, this decay constant was calculated to be −0.101 hr⁻¹.

D. Human Studies

Ten male Veterans attending the medicine outpatient clinic for various ailments underwent a single 24-hour ambulatory blood pressure monitoring to assure normal blood pressure and serum chemistries to assure normal renal function. The average age was 53±14 years, ambulatory BP was 116.7±10.9/72.4±7.1 mm Hg, heart rate was 72.6±9.6, body weight was 90.6±22.4 kg and the average height was 71±3.4 inches. Three individuals were smokers, nine were Caucasian and one was African-American. The concentration of MDA in the plasma of normal volunteers was 0.69±0.13 μmol/L which was similar to the 0.60±0.13 obtained by Wang et al. (11) and Suttnar et al. (12). Urine concentration of MDA in normal volunteers was measured as 1.94±0.79 μmol/g creatinine which was higher than the 0.89±0.35 nmol/mg creatinine reported by Knight et al. in 121 healthy men (13). Obesity, comorbid conditions, diet, smoking, and the older age of the present population may account for the observed differences. However, Guichardant et al. reported MDA levels by a fluorometric HPLC method of 0.23±0.02 μmol/mmol creatinine in healthy volunteers. The present results expressed in the same units were 0.22±0.089 μmol/mmol creatinine (14). Thus, methodologic differences may account for the variability between studies.

Based on the foregoing results, a new method for measuring MDA levels in biological samples has been developed that eliminates extensive column washes, reduced column life and protein precipitation steps. N-butanol was also found to be a suitable extracting solvent. This method is rapid and is reproducible both intraday and between days. However, because MDA degrades approximately 10% per hour, the best performance of the assay requires analyzing samples within an hour of derivatization.

### EXAMPLE II

Detection of Oxidatively Damaged Proteins and Lipids in Urine for Monitoring Oxidative Damage in the Kidney

Angiotensin converting enzyme inhibitors and angiotensin receptor blockers are two classes of drugs that are commonly used for the treatment of progressive kidney disease. However, the optimal dosing for these drugs is not known. The present study demonstrates that monitoring the oxidative damage of urinary proteins and lipids can provide an estimate of oxidative damage in the kidney. Thus, this tool can be used for monitoring the disease state and titrating the dose of these classes of drugs in patients with chronic kidney disease. Previous attempts only study the total protein excretion rate in patients, not the oxidative transformation of such proteins. This is the first report to show that urine protein and lipids can undergo oxidative transformation and that this transformation can be blocked by the angiotensin receptor blocker, losartan.

I. Materials and Methods:

The following materials and methods are provided to facilitate the practice of the present invention.

A. Protocol

The protocol design of this study was reported in detail previously (15). Briefly, patients between the ages of 18 to 80 years with proteinuria of ≧1 g/d, hypertension defined as mean arterial pressure ≧97 mm Hg, serum potassium of ≦5.5 mEq/L and on lisinopril therapy of 40 mg/d for >3 months were eligible for the study. Patients who had previously received angiotensin receptor blockers or with estimated creatine clearance of <30 ml/min were excluded.

A separate group of age matched 10 normotensive volunteers with no history of kidney disease or diabetes served as the control group for plasma and urinary malondialdehyde levels and estimation of plasma protein carbonylation.

The study was a two period, cross-over, randomized controlled trial. Patients received either a sequence of losartan 50 mg/d for 4 weeks, a two week washout, and then placebo for 4 weeks or placebo for 4 weeks, a two week washout, and then losartan 50 mg/d for 4 weeks. Lisinopril 40 mg/d along with other anti-hypertensive therapy were continued throughout the trial. 24-hour urine was collected for protein, sodium, urea and creatinine, urinary carbonylated protein, malondialdehyde and monocyte chemotactic protein-l (MCP1) measurements. As the standard therapy for patients with proteinuria and renal failure includes ACE-inhibitors, ACE-inhibitors were not removed from any patient enrolled in the study.

The study was approved by the Institutional Review Board, and all patients gave written informed consent. Serum chemistries, complete blood counts, urine protein, electrolytes, urea and creatinine were measured in a hospital laboratory using routine methods. Specifically, creatinine was measured on a Hitachi 911 analyzer (Boehringer Mannheim) using the alkaline picrate method and urine protein was measured using a turbidometric method using benzethonium chloride read at 550 nm (Roche Diagnostics Corporation, Indianapolis, Ind.). Ambulatory blood pressure was measured with SpaceLabs 90207 monitors and glomerular filtration rate measurements with continuous infusion of iothalamate as previously reported (15).

B. Plasma and Urinary Malondialdehyde Assays

A rapid and sensitive fluorometric HPLC method was developed for the measurement of malondialdheyde (MDA) in plasma and urine as described in Example I. Briefly, the mobile phase consisted of 40:60 ratio (v/v) of methanol to 50 mM potassium monobasic phosphate at pH 6.8, pumped at a rate of 1.0 ml/min on a Hewlett-Packard Hypersil 5 μ ODS 100×4.6 mm placed in a column warmer set to 37° C. Samples of serum and urine were treated with the antioxidant, butylated hydroxytoluene, and heat derivatized at 100° C. for 1 hour with thiobarbituric acid at an acid pH. Samples were extracted with n-butanol and 10 μl of the extract was injected onto the HPLC apparatus at 1-minute intervals using an autosampler. The Hewlett-Packard model 1046A programmable fluorescence detector was set at excitation of 515 nm and emission of 553 nm. Retention time was 1.87 minutes, however absence of interfering peaks, allowed analysis to be carried out in increments of 1 minute per sample.

C. Total Plasma Protein Carbonyl Measurement

The carbonyl groups, due to oxidative damage of proteins, were detected by derivatizing with dinitrophenyl hydrazine (DNP), separating the derivatizing agent from the proteins and measuring the absorbance at nm. Although the derivatizing agent can be removed with multiple washes of the protein pellet after derivatization, this entails loss of protein during the procedure. Therefore, an HPLC method for measurement of carbonylated proteins has been devised using a size exclusion column to separate the derivatized carbonyl groups from the derivatizing agent and monitoring the separation with a diode array detector. Briefly, the mobile phase consisted of 200 mMol/L sodium monophosphate at pH 6.5 containing 1% SDS pumped at a rate of 1.0 ml/min on an Alltech Macrosphere GPC 7 μ 250×4.6 mm (Alltech Associates, Deerfield, Ill.) placed in a column warmer set to 37° C. Plasma samples were then split in two parts, one was derivatized with 20 mMol/L DNP in 10% Trifluoroacetic acid (TFA) and the other sample was used as a control treated with 10% TFA. Samples were injected using a HP1100 autosampler in a volume of 25 μl of the derivatized and underivatized sample at 8-minute intervals. The Hewlett-Packard model 1100 diode array detector was programmed to retain signals every 2 nm over the 190 nm to 550 nm spectrum and data was recorded using HPLC Chemstation software (Agilent Technologies, Palo Alto, Calif.). The retention time for protein was 3 minutes which was confirmed by a maximum absorbance at 190 nm; the derivatizing agent had a retention time of 7 minutes. The maximum absorbance of DNP was noted at 360 nm. The area under the curve of the 360 nm peak was integrated in the underivatized sample and subtracted from the derivatized sample. A molar extinction coefficient of 22,000 absorbance units/mole was used to determine the concentration of carbonyl in the sample. Data are expressed as nmol of carbonyl per mg protein.

D. Estimation of Carbonylation of Protein in Plasma and Urine Samples by Western Blotting Technique

Oxidation of plasma and urine proteins was measured by Western blot analysis according to the method of Shacter et al. Total protein was determined using the Vitros Dry Slide system (Ortho-Clinical Diagnostics, Rochester, N.Y.). Plasma was diluted 1:25 (v:v) with phosphate-buffered saline (PBS), and one aliquot of the diluted sample was derivatized and another prepared as an underivatized control using the OxyBlot protein oxidation detection kit (Intergen, Purchase, N.Y.). Urine samples were derivatized with DNP or a similar control reagent except that samples were not diluted. Derivatized and underivatized plasma or urine samples were loaded on electrophoresis gels in volumes calculated to give 5 μg of protein per sample, and the samples were electrophoresed according to the method of Laemmli on 4 to 20% gradient SDS-PAGE (Bio-Rad, Hercules, Calif.). Following electroblotting to 0.2 p nitrocellulose for 60 volt hours, the membrane was subsequently blocked using OxyBlot Kit methods and reagents. Bands were visualized with chemiluminescent chemicals and captured on film at two exposure times (30 seconds and 1 minute). Blots were scanned on a Hewlett-Packard ScanJet 5200C scanner (Hewlett-Packard, Palo Alto, Calif.) and analyzed for band area using Un-Scan-It Gel software (Silk Scientific, Orem, Utah).

Samples from individual patients before and after losartan therapy, including derivatized and underivatized controls, were analyzed on a single Western blot. This ensured that the response to losartan therapy was compared under the same analytical conditions. For each plasma or urine sample, carbonyl density was determined from the 30 second exposure which produced clearly visible bands. Density of individual albumin bands and total protein in each sample lane was determined using the same size section of each scanned blot. The analysis box included 26 lanes for each analysis. The uniform window size and analysis box ensured that data were being analyzed consistently from band to band and from blot to blot. Additionally, any density values present in underivatized controls were subtracted from the density of the DNP-treated sample to increase the validity of the comparison among patients.

E. Urinary Monocyte Chemotactic Protein-1 Assay

MCP1 was assayed in urine using a sandwich ELISA (Quantikine® kit for Human MCP1 Immunoassay; R&D Systems, Minneapolis, Minn.). Corrections were made for concentration and values were expressed as ng MCP1 per gm creatinine. A standard curve was generated using a four parameter logistic curve-fit. The correlation coefficient for standards was greater than 0.99 and the lowest detectable limit was 0.7 pg/ml in 1:2 diluted urine. The intra-assay coefficient of variation was 2.5±3.0% and the inter-assay coefficient of variation was 5.6±4.2%.

F. Statistical Analysis

The normality assumption was examined by the Kolmogorov-Smirnov statistic method. Urinary MCP1 and protein excretion were not normally distributed and were log transformed to satisfy the normality assumption. These log-transformed data were used for subsequent analysis. Data were then analyzed by paired t-tests before and after losartan therapy. Results were reported as means ± standard deviation. All tests were two sided at an α level of 0.05. All statistical analysis were carried out using standard procedures on Statistica for Windows (StatSoft, Inc. Tulsa, Okla.) (16).

II. Results:

Sixteen patients (10 African-American, 6 Caucasians; 14 males) of an average age of 53±9 years and body mass index of 38±5.7 kg/M² participated in the trial. Twelve patients had type 2 diabetes mellitus, and the remaining patients suffered from glomerulonephritis. The average seated blood pressure at baseline was 156±18/88±12 mm Hg requiring 3.13±1.2 antihypertensive drugs. Creatinine levels were 2.0±0.8 mg/dL and proteinuria levels were 3.6±0.71 g/g creatinine/24 hours. There was no change in blood pressure or proteinuria in response to add-on losartan therapy as reported previously (15).

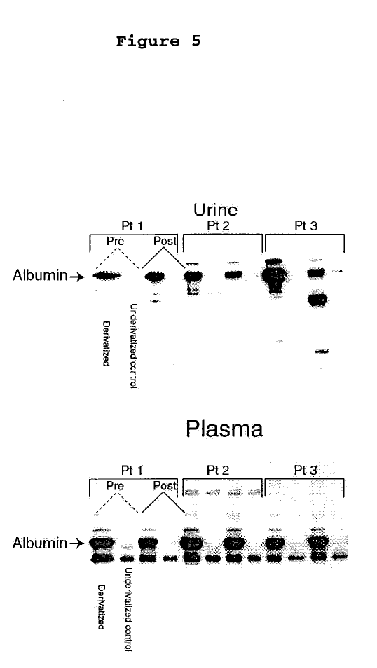

Prior to add-on losartan therapy, urinary protein oxidation was 99% higher than that seen in the plasma (p=0.008). Urinary albumin oxidation was 71% higher than plasma albumin (p=0.045). Oxidized urinary or plasma albumin accounted for the major fraction of total protein oxidation (FIG. 5).

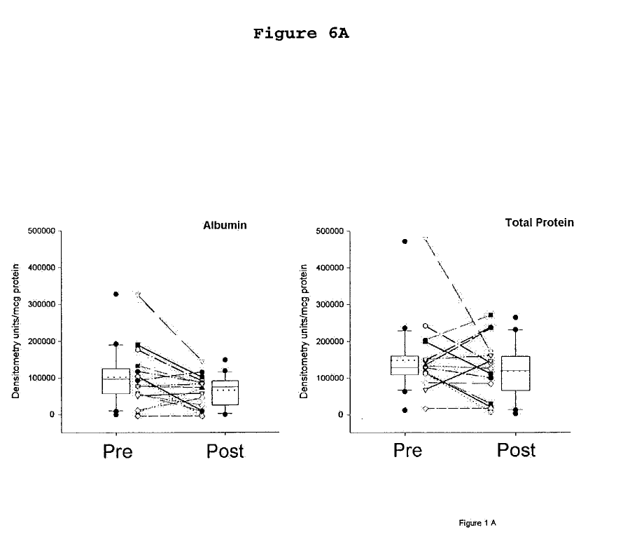

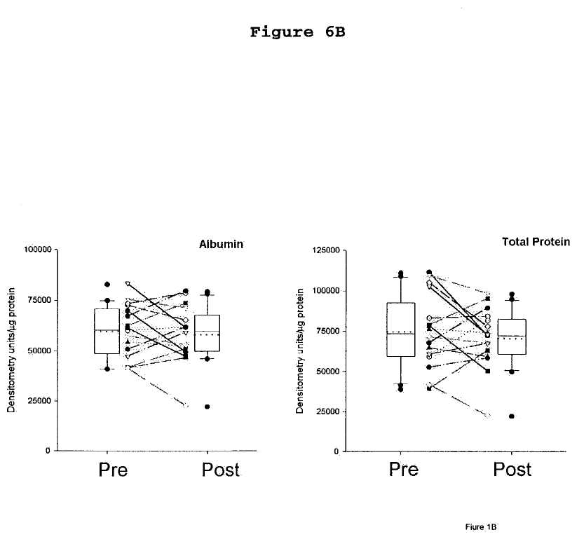

Although proteinuria was not reduced, losartan reduced oxidative damage to urinary albumin (FIG. 6A). This effect was particularly pronounced in those patients who had a high level of oxidized albumin in the urine at baseline. After treatment with losartan, urinary albumin was no more oxidized compared to plasma albumin (p=0.45), but total urinary protein remained 69% more oxidized after losartan therapy (p=0.029). A trend towards improvement in total urinary protein oxidation was also observed but did not reach statistical significance. Plasma albumin and protein oxidation remained unchanged (FIG. 6B).

Samples of urine and plasma malondialdehyde from ten normotensive volunteers, age 53±14 years, were 1.94±0.79 μmol/g creatinine and 0.69±0.13 μmol/L respectively. In comparison, urinary MDA and plasma MDA were elevated in the test patients. However, after losartan therapy, urinary MDA levels significantly decreased from 4.75±3.23 μmol/g creatinine to 3.39±2.17 μmol/g creatinine in the test patients (FIG. 6B).

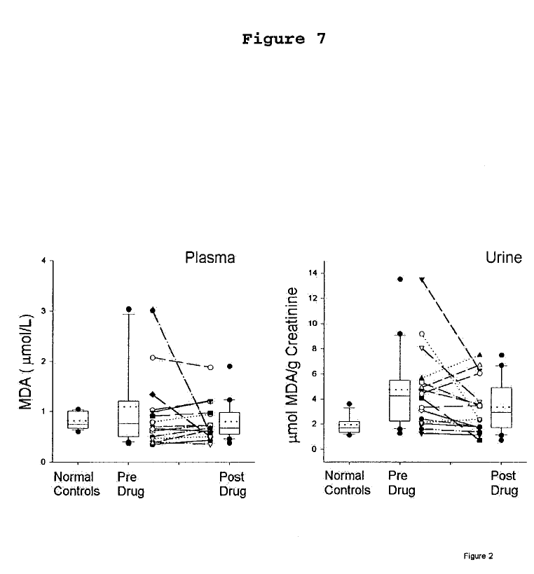

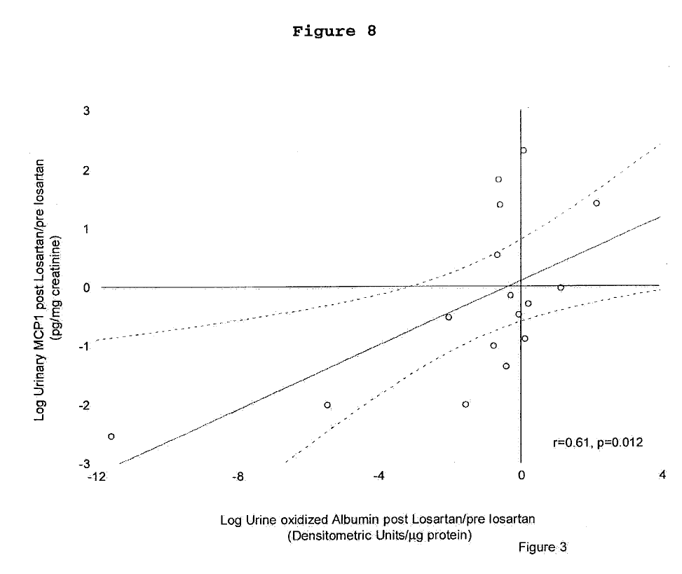

Neither plasma MDA or plasma oxidized proteins (FIGS. 6B and 7) changed in response to losartan therapy. By directly measuring protein carbonylation in plasma, it was further determined that the carbonyl concentration from the oxidized proteins remained unchanged. Finally, there was a good correlation observed between the change in urinary oxidized albumin and urinary MCP-1 levels (FIG. 8).

III. Discussion:

These results demonstrate that the greater proportion of urinary protein oxidation compared to protein oxidation in plasma can be attributed to a number of observations made in cell cultures and animal models. Although vascular superoxide production is increased via NADH/NADPH oxidase via angiotensin II (17), angiotensin II may play a larger role in the kidney due to its effects on superoxide anion production by the mesangial cells and tubular cells (18, 19). In fact, animal models of increased oxidative stress induced by diets deficient in vitamin E and selenium show increased reactive oxygen species (ROS) generation, glomerular and tubular hypertrophy and subsequent injury (20). Thus, the kidney may be particularly susceptible to oxidative stress.

The observation of elevated plasma and urinary excretion of MDA in patients with chronic kidney disease, compared to normal controls is consistent with the data in animals with reduced renal mass who show increased tubular oxygen consumption accompanied by increased MDA per tubule, and increased urinary and plasma levels of MDA (21). The decrease in MDA excretion rate with additional angiotensin II antagonism, despite no change in plasma levels, suggests that the renal generation of MDA, but not the system production of MDA, was reduced. The known pro-oxidant effects of angiotensin II on the kidney lend biologic plausibility to these observations(18, 19).

Stimulation of lipid peroxide production involves protein kinase C (22), an enzyme whose activity is reduced by AT1 receptor antagonism (23). Although the results did not show a correlation in the fall in urinary MDA excretion with a reduction in urinary MCP1, oxidized lipids can increase chemokine expression in monocytes (24) and mesangial cells (25). Therefore, decreases in urinary MDA excretion may contribute to reduced renal inflammation.

Direct measurements of antioxidant enzymes in the kidney such as superoxide dismutase and glutathione peroxidase is increased in ⅚^{th} nephrectomized rats when treated with the ACE inhibitor, enalapril (26). This may account for improvement in the oxidative state in the kidney in preference of the plasma oxidative state.

Although there are a variety of cytokines that can be measured in the urine, MCP1 was selected for several reasons. Urinary excretion of MCP1 correlates with the extent of renal inflammation (28) as well as MCP1 gene expression in the tubules, parietal epithelial cells and infiltrating monocytes (29). ROS generation is involved in MCP-1 gene transcription in response to tissue injury likely via NADPH-oxidase (30). Finally, in animal models of inflammatory kidney disease, administration of AT1 receptor antagonists (31) or genetic absence of the AT1α receptor (30), abrogates the early expression of MCP1 in the glomerulus and the infiltration of monocyte/macrophages. Therefore, it was hypothesized that urinary MCP1 could serve as an important measure of the inflammatory state in the kidney and its reduction would be biologically plausible based on the animal experiments.

In this randomized controlled trial of additional angiotensin II blockade, it was shown that protein in the urine undergoes oxidative damage (urinary albumin was 71% more oxidized compared to plasma albumin). Although such oxidative damage to plasma proteins in patients with chronic kidney disease has previously been reported, it is believed that this is the first demonstration of oxidative damage to urinary proteins in humans. Furthermore, the data demonstrate that oxidative damage to urinary protein and lipids can be reduced with additional angiotensin II blockade. This is particularly notable because the reduction of oxidative stress occurred independent of the reduction in proteinuria or blood pressure, the key mediators of progressive renal damage. Furthermore, there was no change in the markers of protein or lipid damage in the plasma of these patients. Thus, the data are consistent with the hypothesis that the urinary measurements of markers of oxidative damage, both carbonyls and lipid hydroperoxides, are more sensitive than plasma measurements in patients with chronic kidney disease. These observations are further strengthened with the significant association of the change in MCP1 in the urine with oxidized albumin, which supports experimental data in animals that demonstrate the important role of the redox state in the kidney with renal fibrosis and progressive kidney damage.

Thus, monitoring the levels of carbonylated proteins and lipids in urine is an important tool which may be used to advantage for assessing oxidative kidney injury in patients with chronic kidney disease.

### REFERENCES

1. Klahr, S., Levey, A. S., Beck, G. J., Caggiula, A. W., Hunsicker, L., Kusek, J. W., and Striker, G. 1994. The effects of dietary protein restriction and blood-pressure control on the progression of chronic renal disease. Modification of Diet in Renal Disease Study Group. N Engl J Med 330:877-884.

2. Nath, K. A. 1992. Tubulointerstitial changes as a major determinant in the progression of renal damage. Am J Kidney Dis. 20:1-17.

3. Wang, Y., Chen, J., Chen, L., Tay, Y. C., Rangan, G. K., and Harris, D. C. 1997. Induction of monocyte chemoattractant protein-1 in proximal tubule cells by urinary protein. J Am Soc Nephrol 8:1537-1545.

4. Wang, Y., Rangan, G. K., Tay, Y. C., Wang, Y., and Harris, D. C. 1999. Induction of monocyte chemoattractant protein-1 by albumin is mediated by nuclear factor kappaB in proximal tubule cells. J Am Soc Nephrol 10:1204-1213.

5. P. M. Abuja and R. Albertini, Clin Chim. Acta, 306 (2001) 1-17.

6. H. Cai and D. G. Harrison, Circ. Res., 87 (2000) 840-844.

7. Requena, J. R. et al., J. Biochem., 322 (1997) 317-325.

8. D. J. Anderson, Clin Chem, 35 (1989) 2152-2153.

9. H. Esterbauer and K. H. Cheeseman, Methods Enzymol., 186 (1990) 407-421.

10. H. H. Draper and M. Hadley, Methods Enzymol., 186 (1990) 421-431.

11. S. H. Wong, J. A. Knight, S. M. Hopfer, O. Zaharia, C. N. Leach, Jr., and F. W. Sunderman, Jr., Clin Chem., 33 (1987) 214-220.

12. J. Suttnar, L. Masova, and J. E. Dyr, J Chromatogr.B Biomed. Sci Appl., 751 (2001) 193-197.

13. J. A. Knight, S. E. Smith, V. E. Kinder, and R. K. Pieper, Clin Chem., 34 (1988) 1107-1110.

14. M. Guichardant, L. Valette-Talbi, C. Cavadini, G. Crozier, and M. Berger, J Chromatogr.B Biomed.Appl., 655 (1994) 112-116.

15. Agarwal, R. 2001. Role of add-on angiotensin receptor blockade with maximized ACE inhibition. Kidney Int 59:2282-2289.

16. Statistica User Manual. (5.5). 2000. Tulsa, Okla., StatSoft, Inc. 1999. Ref Type: Computer Program

17. Rajagopalan, S., Kurz, S., Munzel, T., Tarpey, M., Freeman, B. A., Griendling, K. K., and Harrison, D. G. 1996. Angiotensin II-mediated hypertension in the rat increases vascular superoxide production via membrane NADH/NADPH oxidase activation. Contribution to alterations of vasomotor tone. J Clin Invest 97:1916-1923.

18. Jaimes, E. A., Galceran, J. M., and Raij, L. 1998. Angiotensin II induces superoxide anion production by mesangial cells. Kidney Int 54:775-784.

19. Hannken, T., Schroeder, R., Stahl, R. A., and Wolf, G. 1998. Angiotensin II-mediated expression of p27Kip1 and induction of cellular hypertrophy in renal tubular cells depend on the generation of oxygen radicals. Kidney Int 54:1923-1933.

20. Nath, K. A. and Salahudeen, A. K. 1990. Induction of renal growth and injury in the intact rat kidney by dietary deficiency of antioxidants. J Clin Invest 86:1179-1192.

21. Nath, K. A., Croatt, A. J., and Hostetter, T. H. 1990. Oxygen consumption and oxidant stress in surviving nephrons. Am J Physiol 258:F1354-F1362.

22. Ha, H. and Endou, H. 1992. Lipid peroxidation in isolated rat nephron segments. Am J Physiol 263:F201-F207.

23. Goodfriend, T. L., Elliott, M. E., and Catt, K. J. 1996. Angiotensin receptors and their antagonists. N. Engl. J Med 334:1649-1654.

24. Terkeltaub, R., Banka, C. L., Solan, J., Santoro, D., Brand, K., and Curtiss, L. K. 1994. Oxidized LDL induces monocytic cell expression of interleukin-8, a chemokine with T-lymphocyte chemotactic activity. Arterioscler.Thromb. 14:47-53.

25. Kamanna, V. S., Pai, R., Roh, D. D., and Kirschenbaum, M. A. 1996. Oxidative modification of low-density lipoprotein enhances the murine mesangial cell cytokines associated with monocyte migration, differentiation, and proliferation. Lab Invest 74:1067-1079.

26. Verbeelen, D. L., De Craemer, D., Peeters, P., Vanden Houte, K., and Van den, B. C. 1998. Enalapril increases antioxidant enzyme activity in renal cortical tissue of five-sixths-nephrectomized rats. Nephron 80:214-219.

27. Rovin, B. H., Doe, N., and Tan, L. C. 1996. Monocyte chemoattractant protein-1 levels in patients with glomerular disease. Am J Kidney Dis 27:640-646.

28. Wada, T., Yokoyama, H., Su, S. B., Mukaida, N., Iwano, M., Dohi, K., Takahashi, Y., Sasaki, T., Furuichi, K., Segawa, C. et al. 1996. Monitoring urinary levels of monocyte chemotactic and activating factor reflects disease activity of lupus nephritis. Kidney Int 49:761-767.

29. Grandaliano, G., Gesualdo, L., Ranieri, E., Monno, R., Montinaro, V., Marra, F., and Schena, F. P. 1996. Monocyte chemotactic peptide-1 expression in acute and chronic human nephritides: a pathogenetic role in interstitial monocytes recruitment. J Am Soc Nephrol 7:906-913.

30. Satriano, J. A., Shuldiner, M., Hora, K., Xing, Y., Shan, Z., and Schlondorff, D. 1993. Oxygen radicals as second messengers for expression of the monocyte chemoattractant protein, JE/MCP-1, and the monocyte colony-stimulating factor, CSF-1, in response to tumor necrosis factor-alpha and immunoglobulin G. Evidence for involvement of reduced nicotinamide adenine dinucleotide phosphate (NADPH)-dependent oxidase. J Clin Invest 92:1564-1571.

31. Wolf, G., Schneider, A., Helmchen, U., and Stahl, R. A. 1998. AT1-receptor antagonists abolish glomerular MCP-1 expression in a model of mesangial proliferative glomerulonephritis. Exp. Nephrol 6:112-120.

While certain of the preferred embodiments of the present invention have been described and specifically exemplified above, it is not intended that the invention be limited to such embodiments. Various modifications may be made thereto without departing from the scope and spirit of the present invention, as set forth in the following claims.

What is claimed is:

1. A method for detecting oxidative kidney injury in an animal, comprising:
a) obtaining a biological sample from an animal suspected of having kidney disease; and
b) detecting oxidatively damaged proteins and lipids in said biological sample, the presence of said oxidatively damaged proteins and lipids being indicative of oxidative kidney injury in said animal.

2. The method of claim 1, wherein said biological sample is urine.

3. The method of claim 1, wherein said oxidatively damaged proteins and lipids are detected using HPLC.

4. The method of claim 1, wherein said oxidatively damaged proteins are carbonylated.

5. The method of claim 1, wherein said lipid is malondialdehyde.

6. The method of claim 1, wherein said animal is a human.

7. A method for detecting oxidative kidney injury in a patient suspected of having kidney disease, comprising:
a) obtaining a urine sample from said patient suspected of having kidney disease; and
b) detecting the presence, if any, of carbonylated proteins in said urine sample.

8. The method of claim 7, wherein the carbonylated proteins are detected in urine using HPLC.

9. A method for detecting oxidative kidney injury in a patient suspected of having kidney disease, comprising:
a) obtaining a urine sample from said patient suspected of having kidney disease; and
b) detecting the presence, if any, of malondialdehyde in said urine sample, the presence of malondialdehyde being indicative of oxidative kidney injury.

10. The method of claim 9, wherein malondialdehyde is detected using HPLC.

11. A method for assessing the efficacy of a therapeutic agent for treating oxidative kidney injury administered to a patient suspected of having kidney disease, comprising:
a) determining the levels of oxidatively damaged proteins and lipids in urine from said patient before treatment with said therapeutic agent;
b) administering said therapeutic agent to said patient;
C) determining the levels of oxidatively damaged proteins and lipids in urine from said patient during treatment with said therapeutic agents; and
d) determining the levels of oxidatively damaged proteins and lipids in urine from said patient after treatment with said therapeutic agents, a change, if any, in the levels of oxidatively damaged proteins and lipids during and after administration of said therapeutic agents being indicative of the efficacy of said therapeutic agents in treating oxidative kidney injury.

12. The method of claim 11, wherein said therapeutic agents are selected from the group consisting of angiotensin converting enzyme inhibitors, angiotensin receptor blockers and losartan.

13. A kit for diagnosing oxidative kidney injury in a patient suspected of having kidney disease, said kit comprising:
a) an instructional material;
b) a tube or vial for obtaining a sample of urine from said patient suspected of having kidney disease;
c) at least one antioxidant reagent;
d) thiobarbituric acid; and
e) phosphoric acid.

14. The kit of claim 13, wherein said at least one antioxidant reagent is selected from the group consisting of butylated hydroxytoluene and dinitrophenyl hydrazine.
